(19) United States
(12) Patent Application Publication
(10) Pub. No.: US 2011/0207199 A1
(43) Pub. Date: Aug. 25, 2011

(54) NOVEL METHOD TO GENERATE MEGANUCLEASES WITH ALTERED CHARACTERISTICS

(75) Inventors: Frederic Paques, Bourg-la-Reine (FR); Sylvestre Grizot, La Garenne Colombes (FR); Philippe Duchateau, Livry Gargan (FR)

(73) Assignee: CELLECTIS, Romainville Cedex (FR)

(21) Appl. No.: 13/057,528

(22) PCT Filed: Feb. 9, 2009
(86) PCT No.: PCT/IB2009/000486

§ 371 Date: May 3, 2011

(30) Foreign Application Priority Data
Aug. 4, 2008 (IB) PCT/IB2008/002999
Dec. 12, 2008 (IB) PCT/IB2008/003744

(51) Int. Cl. C12N 9/16; C40B 10/00

(52) U.S. Cl. 435/196; 506/1

(57) ABSTRACT

Method to generate and select a meganuclease having at least two altered characteristics in comparison to a parent meganuclease, comprising the steps: a. constructing from a parent meganuclease, a first series of variants which differ from said parent meganuclease by at least one acid amino substitution; b. screening the variants from said first series of step a. and selecting those which have a first altered characteristic; c. constructing from the selected variants of step b. a second series of variants having a least one other amino acid substitution; d. screening the variants from said series of step b. and selecting those which have said first altered characteristic and a second altered characteristic. Polypeptide obtained from said method.

The present invention relates to a novel method to engineer and generate meganuclease enzymes with altered specificity for their DNA target, in particular the present invention uses a sequential combinatorial approach to generate engineered meganuclease enzymes.

As increasing amounts of information concerning the genetic nature of disease and related pathologies come to light, new and improved means to engineer a given genetic locus are of particular interest. Among the strategies to engineer a given genetic locus, the use of rare cutting DNA endonucleases such as meganucleases has emerged as a powerful tool to increase the rate of successful gene targeting through the generation of a DNA double strand break (DSB) by a rare cutting DNA endonuclease and a homologous recombination event at the site of the break, so as for instance to replace a defective gene or insert a therapeutic transgene. Meganucleases are endonucleases, which recognize large and hence rare (12-45 bp) DNA target sites. In the wild, meganucleases essentially comprise homing endonucleases, a family of very rare-cutting endonucleases. This enzyme family was first characterized by the use in vivo of the protein I-SceI (Omega nuclease), originally encoded by a mitochondrial group I intron of the yeast Saccharomyces cerevisiae. Homing endonucleases encoded by intron ORFs, independent genes or intervening sequences (inteins) present striking structural and functional properties that distinguish them from “classical” restriction enzymes which generally have been isolated from the bacterial system R/MII.

Homing endonucleases have recognition sequences that span 12-40 by of DNA, whereas “classical” restriction enzymes recognize much shorter stretches of DNA, in the 3-8 by range (up to 12 by for a so called rare-cutter). Therefore homing endonucleases have a very low frequency of cleavage, even in a genome as large and complex as that of a human.

Homing endonucleases fall into four separate families, classified on the basis of conserved amino acids motifs. For review, see Chevalier and Stoddard (Nucleic Acids Research, 2001, 29, 3757-3774).

One of these families and the subject of the present invention is the LAGLIDADG family, the largest of the homing endonucleases families. This family is characterized by a conserved tridimensional structure (see below), but displays very poor conservation at the primary sequence level, except for a short peptide above the catalytic center. This family has been called LAGLIDADG, after a consensus sequence for this peptide, found in one or two copies in each LAGLIDADG protein.

Homing endonucleases with one LAGLIDADG (L) are around 20 kDa in molecular mass and act as homodimers. Those with two copies (LL) range from 25 kDa (230 amino acids) to 50 kDa (HO, 545 amino acids) with between 70 to 150 residues in each motif and act as a monomer. Cleavage of the target sequence occurs inside the recognition site, leaving a 4 nucleotide staggered cut with 3′OH overhangs.

I-CeuI and I-CreI (163 amino acids (SEQ ID NO: 1)) are homing endonucleases with one LAGLIDADG motif (mono-LAGLIDADG). I-DmoI (194 amino acids, SWISSPROT accession number P21505 (SEQ ID NO: 29)), I-SceI, PI-PfuI and PI-SceI are homing endonucleases with two LAGLIDADG motifs.

In the present invention, unless otherwise mentioned, the residue numbers refer to the amino acid numbering of the wild type meganuclease, for instance for I-DmoI sequence SWISSPROT number P21505 (SEQ ID NO: 29) or the structure PDB code 1b24; or for I-CreI the sequence of pdb accession code 1g9y, corresponding to the sequence SEQ ID NO: 1.

Structural models using X-ray crystallography have been generated for I-CreI (PDB code 1g9y), I-DmoI (PDB code 1b24), PI-Sce I, PI-PfuI. Structures of I-CreI and PI-SceI (Moure et al., Nat Struct Biol, 2002, 9: 764-70) bound to their DNA site have also been elucidated leading to a number of predictions about specific protein-DNA contacts.

LAGLIDADG proteins with a single motif, such as I-CreI, form homodimers and cleave palindromic or pseudo-palindromic DNA sequences, whereas the larger, double motif proteins, such as I-SceI are monomers and cleave non-palindromic targets. Several different LAGLIDADG proteins have been crystallized and they exhibit a striking conservation of the core structure that contrasts with a lack of similarity at the primary sequence level (Jurica et al., Mol. Cell. 1998; 2:469-76, Chevalier et al., Nat Struct Biol. 2001; 8:312-6, Chevalier et al., J Mol. Biol. 2003; 329:253-69, Moure et al., J Mol. Biol. 2003; 334:685-95, Moure et al., Nat Struct Biol. 2002; 9:764-70, Ichiyanagi et al., J Mol. Biol. 2000; 300:889-901, Duan et al., Cell. 1997; 89:555-64, Bolduc et al., Genes Dev. 2003; 17:2875-88, Silva et al., J Mol. Biol. 1999; 286:1123-36).

In this core structure, two characteristic αββαββα folds, contributed by two monomers in dimeric LAGLIDADG proteins or by two domains in monomeric LAGLIDADG proteins, face each other with a two-fold symmetry. DNA binding depends on the four β strands from each domain, folded into an antiparallel β-sheet, and forming a saddle on the DNA helix major groove. The catalytic core is central, with a contribution of both symmetric monomers/domains. In addition to this core structure, other domains can be found: for example, PI-SceI, an intein, has a protein splicing domain, and an additional DNA-binding domain (Moure et al., Nat Struct Biol. 2002; 9:764-70, Grindl et al., Nucleic Acids Res. 1998; 26:1857-62).

Despite an apparent lack of sequence conservation between individual members of the LAGLIDADG family, structural comparisons indicate that LAGLIDADG proteins, whether they cut as dimers like I-CreI or as a monomer like I-DmoI, adopt a similar active conformation. In all structures, the LAGLIDADG motifs are central and form two packed α-helices where a 2-fold (pseudo-) symmetry axis separates two monomers or apparent domains.

The LAGLIDADG motif corresponds to residues 13 to 21 in I-CreI, and to positions 14 to 22 and 110 to 118, in I-DmoI. On either side of the LAGLIDADG α-helices, a four β-sheet provides a DNA binding interface that drives the interaction of the protein with the half site of the target DNA sequence. I-DmoI is similar to I-CreI dimers, except that the first domain (residues 1 to 95) and the second domain (residues 105 to 194) are separated by a linker (residues 96 to 104) (Epinat et al., Nucleic Acids Res, 2003, 31: 2952-62).

I-SceI was the first homing endonuclease used to stimulate homologous recombination in mammalian cells, wherein it did so to over 1000-fold at a genomic target (Choulika et al., Mol Cell Biol. 1995; 15:1968-73, Cohen-Tannoudji et al., Mol Cell Biol. 1998; 18:1444-8, Donoho et al., Mol Cell Biol. 1998; 18:4070-8, Alwin et al., Mol. Ther. 2005; 12:610-7, Porteus., Mol. Ther. 2006; 13:438-46, Rouet et al., Mol Cell Biol. 1994; 14:8096-106).

I-SceI has also been used to stimulate targeted recombination in the mouse liver in vivo where recombination could be observed in up to 1% of hepatocytes (Gouble et al., J Gene Med. 2006; 8:616-22). An inherent limitation of such a methodology is that it requires the prior introduction of the natural I-SceI cleavage site into the locus of interest.

To circumvent this limitation, significant efforts have been made over the past years to generate homing endonucleases with tailored cleavage specificities. Given their high level of specificity, homing endonucleases represent ideal scaffolds for engineering tailored endonucleases and several studies have shown that the DNA binding domain from LAGLIDADG proteins, (Chevalier et al., Nucleic Acids Res. 2001; 29:3757-74) can be engineered.

Several LAGLIDADG proteins, including PI-SceI (Gimble et al., J Mol Biol. 2003; 334:993-1008), I-CreI (Seligman et al., Nucleic Acids Res. 2002; 30:3870-9, Sussman et al., J Mol. Biol. 2004; 342:31-41, Rosen et al., Nucleic Acids Res. 2006; Arnould et al., J Mol. Biol. 2006; 355:443-58), I-SceI (Doyon et al., J Am Chem Soc. 2006; 128:2477-84) and I-MsoI (Ashworth et al., Nature. 2006; 441:656-9) have been modified by rational or semi-rational mutagenesis and screening to acquire new sequence binding or cleavage specificities.

Several different groups, including the inventors, have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

Hundreds of I-CreI derivatives with locally altered specificity have been engineered by combining the semi-rational approach and High Throughput Screening:
Residues Q44, R68 and R70 or Q44, R68, D75 and 177 of I-CreI were mutagenized and a collection of variants with altered specificity at positions ±3 to 5 of the I-CreI DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38, or N30, Y33 and Q38 or K28, Y33, Q38 and S40 of I-CreI were mutagenized and a collection of variants with altered specificity at positions ±8 to 10 of the I-CreI DNA target (10NNN DNA target) were identified by screening (Smith et al., Nucleic Acids Res., 2006, 34, e149; International PCT Applications WO 2007/060495 and WO 2007/049156).

To further increase the range of targets which I-CreI can target two different variants each comprising a set of mutations have been combined and assembled in a functional heterodimeric endonuclease able to cleave a chimeric target resulting from the fusion of a different half of each variant DNA target sequence (International PCT Applications WO 2006/097854 and WO 2007/034262).

Furthermore the inventors have shown that residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/049095 and WO 2007/057781).

The meganuclease variants obtained with said semi-rational approach and high throughout screening have altered specificity and cleave new DNA targets; however, even though said approach works well, certain DNA targets remain difficult to generate altered meganucleases for.

Seeing the problems with prior art methods of creating meganucleases with various combinations of altered specificity and other properties, the inventors have developed a new method which addresses the limitations of the prior art. In particular the inventors have developed a method based upon the sequential selection and combination of mutations which allows a meganuclease to be altered in the desired way.

In particular instead of combining two mutations sets like in prior art methods, the concept of the sequential combinatorial approach is to fix one mutation set before looking for a further mutation set(s) using the first fixed mutation set as the basis for the subsequent selection.

According to a first aspect of the present invention there is provided a method to generate and select a meganuclease having at least two altered characteristics in comparison to a parent meganuclease, comprising the steps:

a. constructing from a parent meganuclease, a first series of variants which differ from said parent meganuclease by at least one acid amino substitution;

b. screening the variants from said first series of step a. and selecting those which have a first altered characteristic;

c. constructing from the selected variants of step b. a second series of variants having a least one other amino acid substitution;

d. screening the variants from said series of step b. and selecting those which have said first altered characteristic and a second altered characteristic.

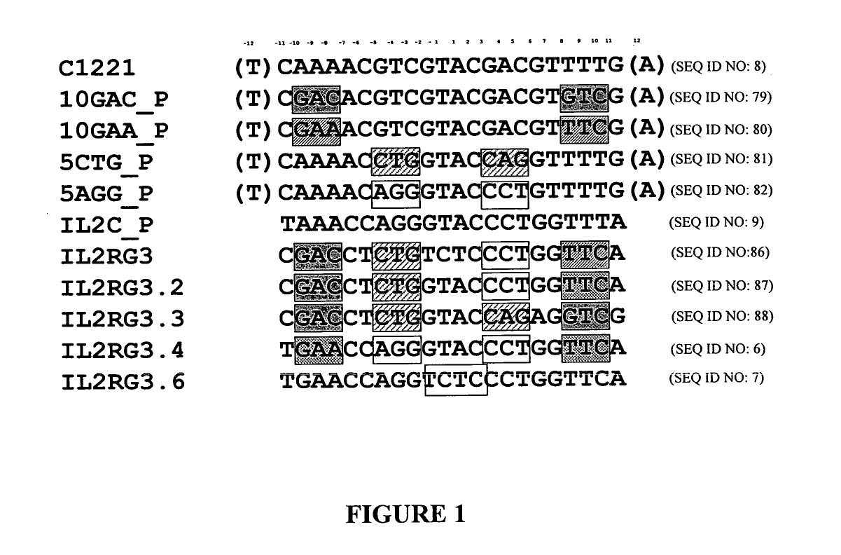

Throughout the present patent application a number of terms and features are used to present and describe the present invention, to clarify the meaning of these terms a number of definitions are set out below and wherein a feature or term is not otherwise specifically defined or obvious from its context the following definitions apply.
Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Cleavage activity: the cleavage activity of the variant according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector. Usually, the genomic DNA target sequence comprises one different half of each (palindromic or pseudo-palindromic) parent homodimeric meganuclease target sequence. Expression of the heterodimeric variant results in a functional endonuclease which is able to cleave the genomic DNA target sequence. This cleavage induces homologous recombination between the direct repeats, resulting in a functional reporter gene (LacZ, for example), whose expression can be monitored by an appropriate assay. The specificity of the cleavage by the variant may be assessed by comparing the cleavage of the (non-palindromic) DNA target sequence with that of the two palindromic sequences cleaved by the parent homodimeric meganucleases or compared with wild type meganuclease.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.
by “meganuclease domain” is intended the region which interacts with one half of the DNA target of a meganuclease and is able to associate with the other domain of the same meganuclease which interacts with the other half of the DNA target to form a functional meganuclease able to cleave said DNA target.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step a. or the meganuclease from which the second series of variants are derived in step c.
by “meganuclease variant” or “variant” is intented a meganuclease obtained by replacement of at least one residue in the amino acid sequence of the parent meganuclease (natural or variant meganuclease) with a different amino acid.
by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “first/second series of variants” it is intended a collection of variant meganucleases, each of which comprises one or more amino acid substitution in comparison to a parent meganuclease from which all the variants in the series are derived.
by “first/second altered characteristic” it is intended to mean a measurable or observable characteristic of a meganuclease enzyme which can be compared and hence determined to be altered in comparison to another meganuclease for the same characteristic. Examples of characterisitics include DNA target specificity, activity and enzymatic structure.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “I-CreI” is intended the wild-type I-CreI having the sequence of PDB accession code 1g9y corresponding to the sequence SEQ ID NO:1 in the sequence listing.
by “I-CreI monomer” is intended the full-length wild-type I-CreI amino acid sequence SEQ ID NO: 1 (163 amino acids) or a functional variant thereof comprising amino acid substitutions in SEQ ID NO: 1. I-CreI functions as a dimer, which is made of two I-CreI monomers.
by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application several of the I-CreI variants described comprise an additional Alanine after the first Methionine of the wild type I-CreI sequence. These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” is intended a 22 to 24 by double-stranded DNA sequence which is cleaved by I-CreI. I-CreI sites include the wild-type (natural) non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221 (FIG. 1).
by “domain” or “core domain” is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises tour beta-strands (β₁β₂β₃β₄) folded in an antiparallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the homing endonuclease I-CreI (163 amino acids) which acts as a dimer, the LAGLIDADG homing endonuclease core domain corresponds to the residues 6 to 94.
by “subdomain” is intended the region of a LAGLIDADG homing endonuclease core domain which interacts with a distinct part of a homing endo-nuclease DNA target half-site.
by “beta-hairpin” is intended two consecutive beta-strands of the antiparallel beta-sheet of a LAGLIDADG homing endonuclease core domain ((β₁β₂ or, β₃β₄) which are connected by a loop or a turn,
by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “DNA target”, “DNA target sequence”, “target sequence”, “target-site”, “target”, “site”; “site of interest”; “recognition site”, “recognition sequence”, “homing recognition site”, “homing site”, “cleavage site” is intended a 20 to 24 by double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated for C1221 (see FIG. 1). Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.
by “DNA target half-site”, “half cleavage site” or half-site” is intended the portion of the DNA target which is bound by each LAGLIDADG homing endonuclease core domain.
by “chimeric DNA target” or “hybrid DNA target” is intended the fusion of a different half of two parent meganuclease target sequences. In addition at least one half of said target may comprise the combination of nucleotides which are bound by at least two separate subdomains (combined DNA target).
by “human IL2RG gene” is intended the normal (wild-type) IL2RG located on chromosome X (Xq13.1; Gene ID: 3561) and the mutated IL2RG genes (mutant IL2RG; IL2RG allele), in particular the mutants responsible for SCID-X1. The human IL2RG gene (4145 bp) corresponds to positions 70243984 to 70248128 on the reverse complement strand of the sequence accession number GenBank NC_{—}000023.9. It comprises eight exons (Exon 1: positions 1 to 129; Exon 2: positions 504 to 657; Exon 3: positions 866 to 1050; Exon 4: positions 1259 to 1398; Exon 5: positions 2164 to 2326; Exon 6: positions 2859 to 2955; Exon 7: positions 3208 to 3277; Exon 8: positions 3633 to 4145). The ORF which is from position 15 (Exon 1) to position 3818 (Exon 8), is flanked by short and long untranslated regions, respectively at the 5′ and 3′ end. The wild-type IL2RG gene sequence corresponds to SEQ ID NO: 3 in the sequence listing; the mRNA sequence corresponds to GenBank NM_{—}000206 (SEQ ID NO: 4) and the gamma C receptor amino acid sequence to GenBank NP_{—}000197 (SEQ ID NO: 5). The mature protein (347 amino acids) is derived from a 369 amino acid precursor comprising a 22 amino acid N-terminal signal peptide.
by “DNA target sequence from the IL2RG gene” it is intended a 20 to 24 by sequence of a primate (simian) IL2RG gene locus, for example the human IL2RG gene locus, which is recognized and cleaved by a meganuclease variant or a single-chain chimeric meganuclease derivative.
by “beta-2-microglobulin gene” is intended the beta-2-microglobulin gene of a mammal. For example, the human beta-2-microglobulin gene (B2M, 6673 bp (SEQ ID NO: 83) is situated from positions 42790977 to 42797649 of the sequence corresponding to accession number NC_{—}000015. The B2M gene comprises four exons (Exon 1: positions 1-127; Exon 2: positions 3937 to 4215; Exon 3: 4843 to 4870; Exon 4: positions 6121 to 6673). The ORF which is from position 61 (Exon 1) to positions 4856 (Exon 3), is flanked by a short and a long untranslated region, respectively at its 5′ and 3′ ends.
by “DNA target sequence from the beta-2-microglobulin gene” it is intended a 20 to 24 by sequence of the beta-2-microglobulin gene of a mammal which is recognized and cleaved by a meganuclease variant.
by “vector” is intended a nucleic acid molecule capable of trans-porting another nucleic acid to which it has been linked.
by “homologous” is intended a sequence with enough identity to another one to lead to a homologous recombination between sequences, more particularly having at least 95% identity, preferably 97% identity and more preferably 99%.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

This new method has a number of advantages over prior art methods and in particular a major advantage of the method according to the present invention is that synergistic mutations can be found, these are mutations which do not generate the desired characteristic by themselves but instead act with other mutations to elicit the desired characteristic. Such synergistic mutations cannot be found using prior art methods, as the selection at different times of the two or more desired characteristics means that mutations which give rise to both characterisitics simultaneously are not selected for and hence cannot be found.

A further advantage of this method is that it can be used to generate meganuclease enzymes to targets for which previous attempts with prior art methods have failed. Examples of this are set out in the detailed description below.

In particular this method is useful for generating and selected an altered meganuclease, starting with a parent meganuclease which is a member from the LAGLIDADG family.

In particular this method involves the construction in steps a. and c. of a first and a second series of variants which differ from their respective parent meganuclease by at least one amino acid substitution in at least one of the functional domains or subdomains of said first and/or second series of variants. In particular the parent meganuclease is either a wildtype meganuclease or a functional variant of a wild type meganuclease.

In particular the selected meganuclease from step a. or c. is a single-chain meganuclease.

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric mega-nucleases for use either as the parent meganuclease in the method according to the present invention or so as to combine two monomers from the same or different mega-nucleases generated using the present method into a single chain chimeric mega-nuclease. In addition such methods can also be used to convert any of the specific variants detailed in the present patent application into a single chain meganuclease. In particular the parent meganuclease is selected from the group comprising: I-Sce I, I-Chu I, I-Cre I, I-Csm I, PI-Sce I, PI-Tli I, PI-Mtu I, I-Ceu I, I-SceII, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka 1, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I, I-MsoI, I-DmoI.

The inventors have shown that their new sequential combinatorial method works with a number of meganucleases and therefore this same method can be used to engineer the characteristics of any other meganuclease selected from the above list. This list is not exhaustive and other meganuclease are encompassed by the present invention as are known variants of for instance I-CreI, I-DmoI and I-MsoI.

In particular the parent meganuclease comprises at least one I-CreI monomer.

I-CreI is amongst the most studied and characterised of all the meganucleases. Extensive structural and biochemical information is available for this enzyme as are a vast array of existing variants which show various characterisitics. Therefore using this existing store of information, a meganuclease comprising one I-CreI monomer such as a chimeric fusion protein or a homo or hetero-dimeric I-CreI enzyme, is a preferred starting point for the method according to the present invention.

In particular the at least one I-CreI monomer is modified in step a. and/or step c. of said method, such that at least one of the residues in positions 19, 24, 28, 30, 32, 33, 37, 38, 40, 44, 50, 54, 66, 68, 70, 75, 77, 79, 80, 81, 105, 129, 132 of said I-CreI monomer is substituted.

The above list is not exhaustive and any of the 163 amino acid residues in I-CreI can be altered either by random mutagenesis or site directed mutagenesis in accordance with the present invention.

Previous work with I-CreI has identified a number of residues which when substituted can affect the target specificity of the enzyme.

In a preferred embodiment of the present invention therefore, the substitution of at least one of these residues in step a. and/or step c. can be used as the starting point for the present method.

In particular the parent meganuclease is chimeric comprising a first domain from a first meganuclease and a second domain from a second meganuclease.

As well as seeking to broaden the target of a meganuclease by altering one or more residues in the enzymatic domain of a single meganuclease, workers have also combined enzymatic domains from different meganucleases, such as E-DreI (Chevalier et al, Mol. Cell. 2002; 10:895-905). E-DreI consists of the fusion of the N-terminal domain of I-DmoI to a single subunit of the I-CreI homodimer linked by a flexible linker to create the initial scaffold for the enzyme.

The inventors have also created several I-CreI/I-DmoI hybrids called DmoCre (Smith J et al., Nucleic Acids Res. 2006; 34(22):e149, Arnould S et al., J Mol Biol. 2006; 355:443-58, Arnould S et al., J Mol Biol. 2007; 371:49-65), with later versions called DmoCre2 (SEQ ID NO: 36) and DmoCre4 (SEQ ID NO: 104). Such chimeric meganucleases can also be used as the parent meganuclease in the method according to the present invention.

According to this aspect of the present invention the first domain and/or second domain can comprise in the case of a dimeric meganuclease, the complete monomer for instance of I-CreI. Or in the case of a monomeric enzyme such as I-DmoI, the first and/or second domain can comprise a portion of the enzyme, this portion comprising the essential enzymatic domains, as discussed above, so as to allow the chimeric enzyme to function.

In particular the first domain is from I-DmoI.

In accordance with this aspect of the present invention the I-DmoI domain consists of residues 1 to 95 of the wild type I-DmoI protein (SEQ ID NO: 29). In particular I-DmoI domain may also comprise the I-DmoI linker, located at positions 96 to 104 and the beginning of the second I-DmoI domain located at positions 105 to 109 of the wild type I-DmoI protein (SEQ ID NO: 29).

In particular this method involves the selection of at least one meganuclease which has at least two altered characteristics which are selected from the group comprising: altered DNA target specificity for at least one nucleotide in said DNA target; altered enzymatic activity levels; altered kinetics; altered domain-domain structure.

In addition to the generation of meganucleases with altered target specificity, this new sequential combinatorial method can also be used to select for other alterations in meganuclease activity such as increased activity, activity at a selected temperature for instance 37° C. or the selection of a meganuclease which has more stable domain—domain structures either within a monomeric or hetero/homodimeric meganuclease.

In particular according to the present method, the first series of variants and/or the second series of variants are obtained by constructing a nucleic acid library encoding said parent meganuclease of step a. or encoding the selected variants of step b. respectively; and
mutating said nucleic acid libraries so as to introduce a mutation into the sequence encoded therein; and
expressing the first series of variants and/or the second series of variants from each of said respective libraries for screening in steps b. and d. respectively.

In particular one or both of the libraries of nucleic acid molecules are created by random mutagenesis of a nucleic acid molecule encoding said parent meganuclease from step a. or encoding the selected variants of step b.

Random mutagenesis of the coding sequences of the meganuclease forms one aspect of the present invention. Random mutagenesis is attractive as it is possible to obtain unexpected mutations which have characteristic altering properties. Conversely, random mutagenesis requires that a larger pool of mutants to be sampled as on a per mutation basis the chance of obtaining a valuable mutant is smaller than using a site-directed mutagenesis approach.

Alternatively one or both of the libraries of nucleic acid molecules are created by site directed mutagenesis.

As discussed above site directed mutagenesis has previously been used in the re-engineering of meganucleases, for instance specific mutations of an amino acid residue thought or known to contact a particular nucleotide often lead to an alteration in specificity of the mutant meganuclease for this particular nucleotide. Site directed mutagenesis can therefore be used to increase the chances that a desired alteration will occur in the mutant library.

In addition to purely random or site directed mutagenesis regimes, the present invention also encompasses methods involving a combination of these two approaches, that is a method involving the site directed mutagenesis of one or more selected amino acid residues as well as a background level of mutagenesis across all the residues of the meganuclease.

In particular steps c. and d. of the method can be repeated a number of times (‘n’) to generate a meganuclease with a number of additional altered characteristic(s) (‘n’).

The present invention relates to a method to generate a meganuclease which has at least two altered characteristics in comparison to the parent meganuclease. The present invention can also however be used to generate a mega-nuclease which comprises additional altered characteristics. To do this steps c. and d. of the method are repeated and the selection of meganuclease mutants showing the required combination of altered characteristics are made in each iteration of step d. Steps c. and d. can be repeated a number of times ‘n’ so as to generate a meganuclease with a number of altered characteristics n (plus the original two altered characteristics).

In particular the I-DmoI domain is modified in step a. and/or step c. of said method, such that at least one of residues in position 4, 15, 19, 20 27, 29, 33, 35, 37, 49, 52, 75, 76, 77, 81, 92, 94, 95 101, 102, and/or 109 of said first I-DmoI domain.

The present invention also relates to a polypeptide which comprises or consists of any one of SEQ ID NO: 51, 52, 53, 54, 55, 56, 57, 58, 59, 60, 61, 62, 63, 64, 65, 66, 67, 68, 69, 70, 71, 72, 73, 74, 75, 76, 77, 78, 89, 90, 91, 92, 93, 94, 94, 95, 96, 97, 105, 106, 107, 108, 109, 110, 112; said polypeptide being able to be obtained according to the method defined above.

In particular the polypeptide is a functional meganuclease in vitro and in vivo.

In particular the polypeptide is an I-CreI variant.

In particular, the polypeptide according to this aspect of the present invention may comprise a detectable tag at its NH₂ and/or COOH terminus.

The present invention also relates to a polynucleotide, this polynucleotide being characterized in that it encodes a polypeptide according to the present invention.

The present invention also relates to a vector, characterized in that it comprises a polynucleotide according to the present invention.

The present invention also relates to a host cell, characterized in that it is modified by a polynucleotide or a vector according to the present invention.

The recombinant vectors comprising said polynucleotide may be obtained and introduced in a host cell by the well-known recombinant DNA and genetic engineering techniques.

The polypeptide of the invention may be obtained by culturing the host cell containing an expression vector comprising a polynucleotide sequence encoding said polypeptide, under conditions suitable for the expression of the polypeptide, and recovering the polypeptide from the host cell culture.

The present invention also relates to a non-human transgenic animal, characterized in that all or part of its constituent cells is modified by a polynucleotide or a vector according to the present invention.

The present invention also relates to a transgenic plant, characterized in that all or part of its constituent cells is modified by a polynucleotide or a vector according to the present invention.

The present invention also relates to the use of a meganuclease according to the present invention in a therapeutic method, in particular a meganuclease according to the present invention can be used for genome therapy ex vivo (gene cell therapy) and genome engineering. Most particularly the described meganucleases could be used to insert, delete or repair an endogenous or exogenous coding sequence.

To do this the meganuclease (or a polynucleotide encoding said meganuclease) and/or the targeting DNA are contained within a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding the meganuclease are imported or translocated by the cell from the cytoplasm to the site of action in the nucleus. Whilst within the nucleus the meganuclease will cut any targets present in the genome and the vector resulting in double strand breaks which will be repaired by the endogenous repair mechanisms of the host cell and when a repair occurs between the genome and vector sequence this will result in a genome engineering event such as an insertion, deletion or repair.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

For a better understanding of the invention and to show how the same may be carried into effect, there will now be shown by way of example only, specific embodiments, methods and processes according to the present invention with reference to the accompanying drawings in which:

FIG. 1: represents the IL2RG3 target sequences and its derivatives. All targets are aligned with the C1221 target (SEQ ID NO: 8), a palindromic sequence cleaved by I-CreI. 10GAC_P, 10GAA_P, 5CTG_P and 5AGG_P (SEQ ID NO: 79-82) are close derivatives found to be cleaved by I-CreI mutants. They differ from C1221 by the boxed motives. IL2C_P (SEQ ID NO: 9) differs from 5AGG_P by the bases at position ±11 and ±7. The IL2RG3.6 target (SEQ ID NO: 7) differs from IL2RG3.4 by the boxed four central bases. C1221, 10GAC_P, 10GAA_P, 5CTG_P and 5AGG_P were first described as 24 by sequences, but structural data suggest that only the 22 by are relevant for protein/DNA interaction. However, positions ±12 are indicated in parenthesis. IL2RG3 (SEQ ID NO: 86) is the DNA sequence located in the human IL2RG gene at position 1686. In the IL2RG3.2 target (SEQ ID NO: 87), the TCTC sequence in the middle of the target is replaced with GTAC, the bases found in C1221. IL2RG3.3 (SEQ ID NO: 88) is the palindromic sequence derived from the left part of IL2RG3.2, and IL2RG3.4 (SEQ ID NO: 6) is the palindromic sequence derived from the right part of IL2RG3.2. As shown in the Figure, the boxed motives from 10GAC_P, 10GAA_P, 5CTG_P and 5AGG_P are found in the IL2RG3 series of targets.

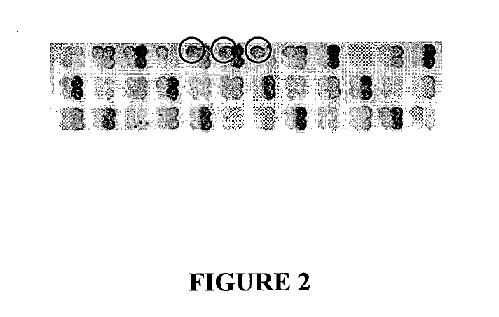

FIG. 2: represents yeast screening of 5AGG_P cutters against the IL2C_P target. Mutants are in the upper left dot of the cluster. The two right dots are experiment internal controls. The three clones that were chosen for further studies are circled.

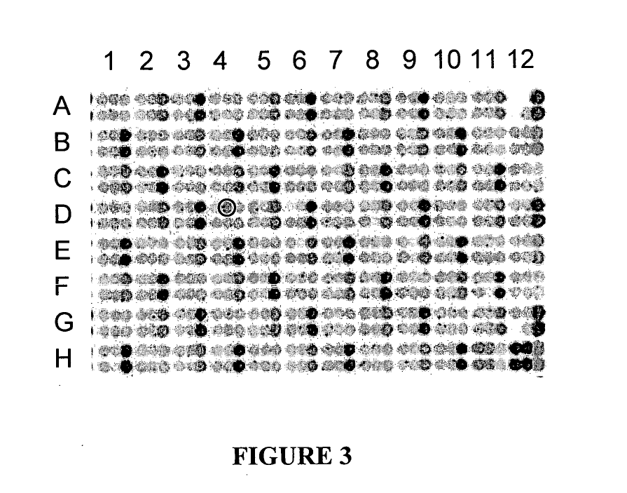

FIG. 3: represents an example of primary screening of mutants belonging to the SeqLib1 library against the IL2RG3.4 target (SEQ ID NO:6). Columns and rows are respectively noted from 1 to 12 and from A to H. In each 6 dots yeast cluster, four SeqLib1 mutants are screened against the IL2RG3.4 target. The two right dots are cluster internal controls. H10, H11 and H12 are also experiment controls. A positive clone is circled.

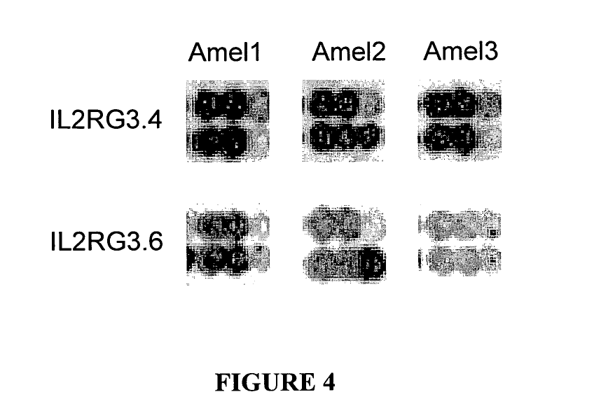

FIG. 4: represents cleavage activity of the three mutants Amell to Amel3 (SEQ ID NO:26 to 28) toward the IL2RG3.4 (SEQ ID NO:6) and IL2RG3.6 (SEQ ID NO:7) targets. In each 6 dots yeast cluster, the same mutant is screened four times against the same target (four left dots). The upper right dot is the Seq4 mutant and the bottom right dot is an experiment internal control.

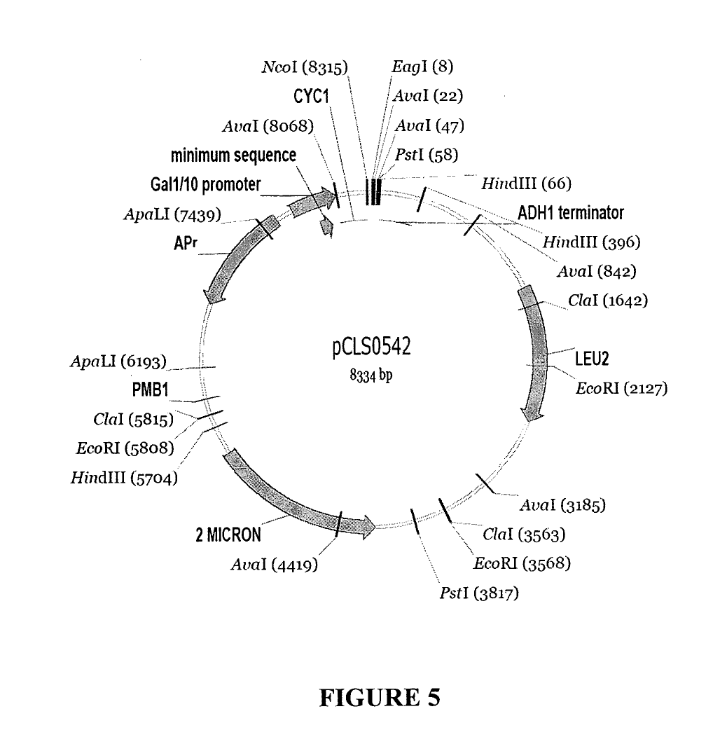

FIG. 5: Schematic Restriction map of pCLS0542

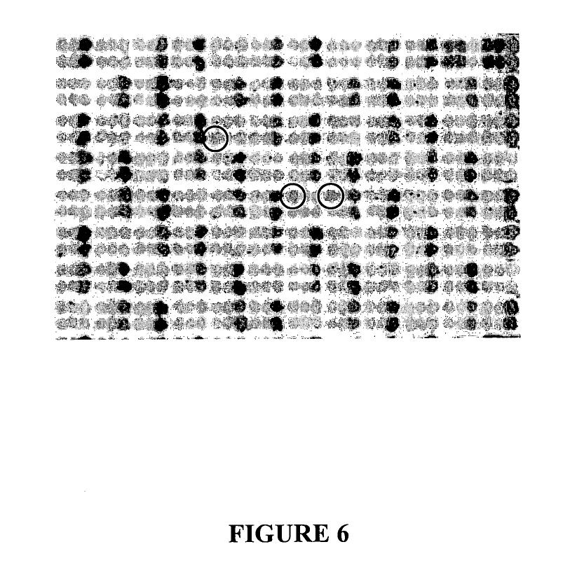

FIG. 6: The figure displays an example of primary screening of DmoCre2 mutants from the SeqDC10NNN4ACT library against the combined DC10TGG4ACT target. In each yeast cluster, the two right dots are experiment internal controls. For the other four dots, one dot corresponds to one mutant from the SeqDC10NNN4ACT library. Three positives clones are black circled.

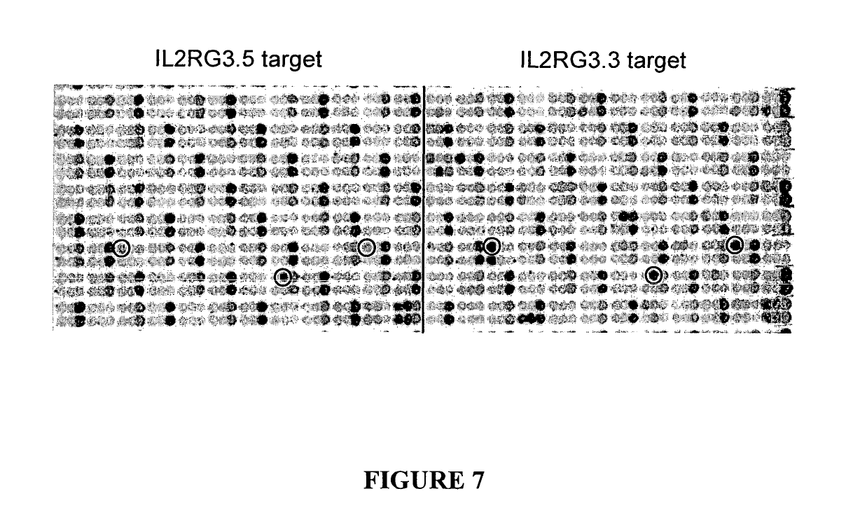

FIG. 7: Example of primary screening of the SeqIL2RG3-2 mutant library toward both IL2RG3.3 (SEQ ID NO:88) and IL2RG3.5 targets. In each 6 dots yeast cluster, the two right dots are experiment internal controls while each other dot is a mutant from the library. Some very strong IL2RG3.3 cutters that cleave also the IL2RG3.5 target have been black circled.

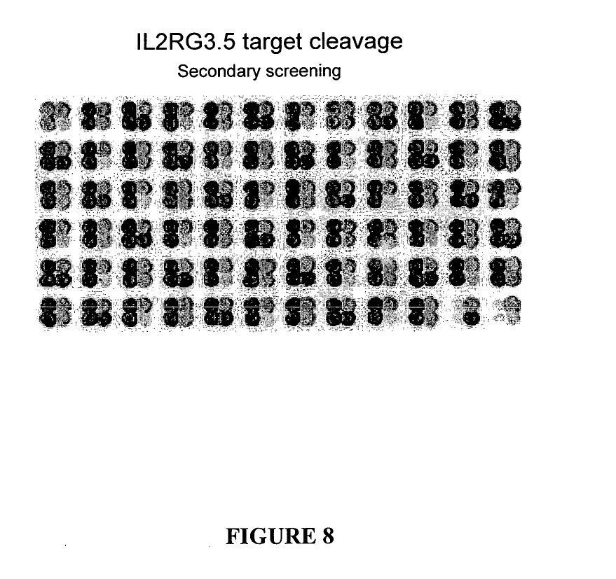

FIG. 8: The figure displays the secondary screening of the improved IL2RG3.5 cutters after addition of single mutation by site directed mutagenesis. In each four dots yeast cluster, the two left dots are an improved IL2RG3.5 mutant, the right bottom dot is an experiment internal control and the upper right dot is the SeqC mutant.

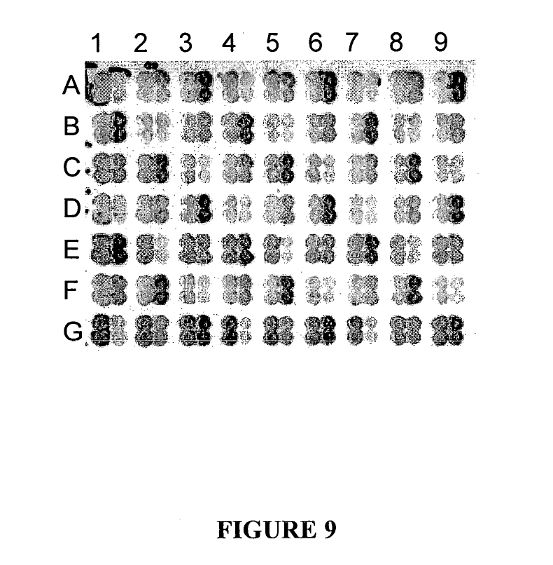

FIG. 9: Yeast screening of heterodimers resulting from coexpression of mutants obtained by the sequential combinatorial method. The 112RG3.4 mutants are in lines A to G (mutants SeqIL2RG4-1 to IL2RG4-7 (SEQ ID NO:72 to 78)) and the IL2RG3.5 mutants are in columns 1 to 9 (mutants SeqIL2RG5-1 to SeqIL2RG5-9 (SEQ ID NO:63 to 71)). In each four dots yeast cluster, the two left dots are a heterodimer and the two right dots, experiment internal controls.

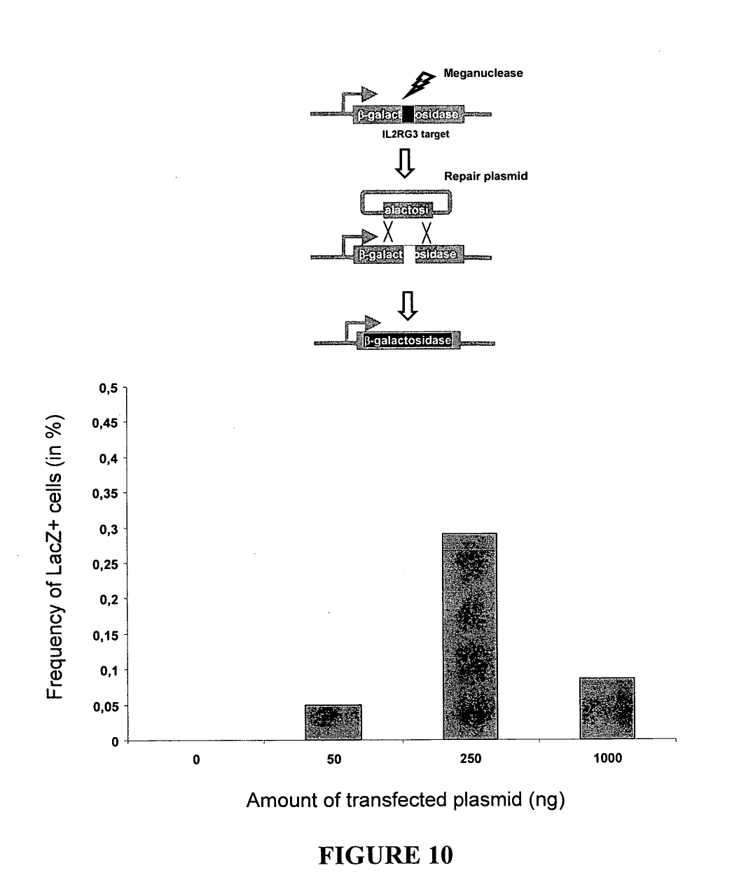

FIG. 10: Gene correction activity of the IL2RG3 meganuclease composed of the two SeqIL2RG5-1 and SeqIL2RG4-7 mutants. The frequency of LacZ positive cells is represented in function of the amount of transfected expression plasmid (for example, 250 ng represents 250 ng of each expression vector).

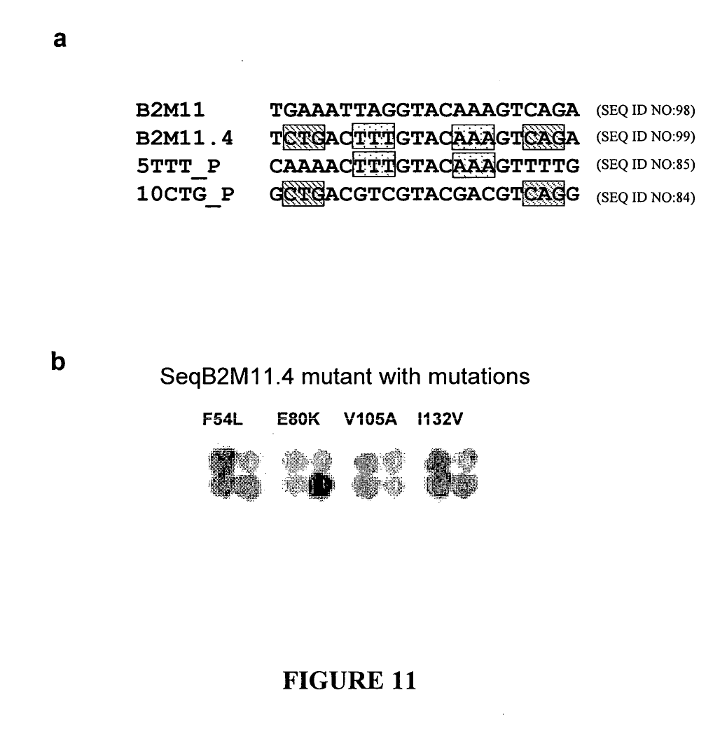

FIG. 11: a. Some 22 bp DNA targets are represented. As shown in the Figure, the boxed motifs from 10CTG_P and 5TTT_P are found in the B2M11.4 target. b. Yeast screening toward the B2M11.4 target of the SeqB2M11.4 mutant carrying single mutations. In each four dots yeast cluster, the two left dots are the mutant, the bottom right dot is an experiment internal control and the upper right dot is the SeqB2M11.4 mutant (cleavage almost not detectable in this experiment).

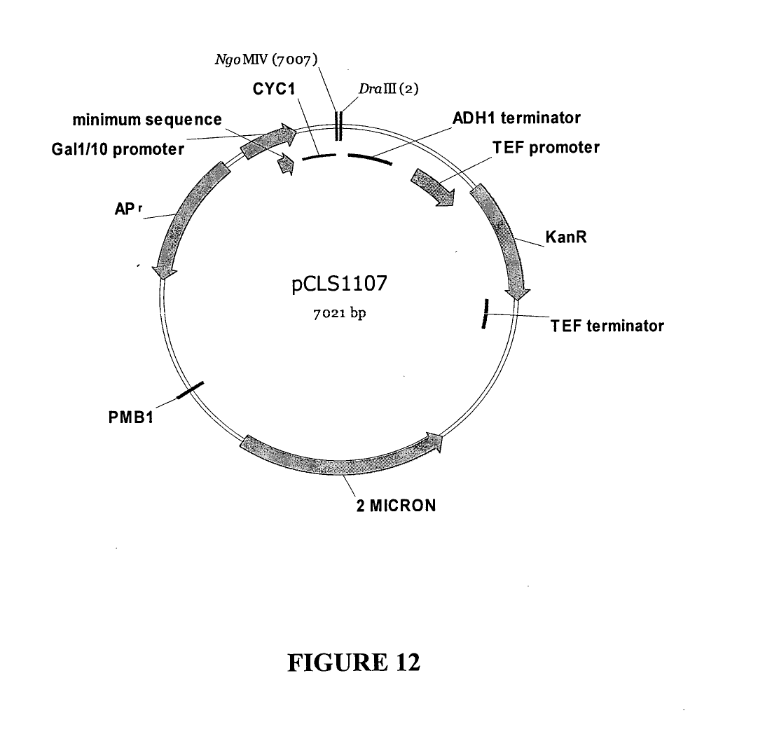

FIG. 12: represents the pCLS1107 vector map.

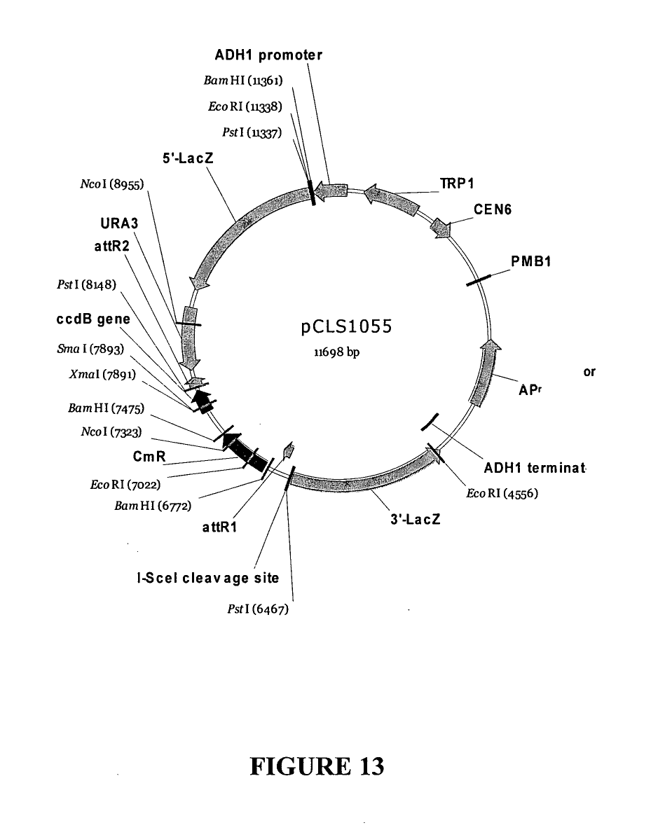

FIG. 13: represents the pCLS1055 vector map.

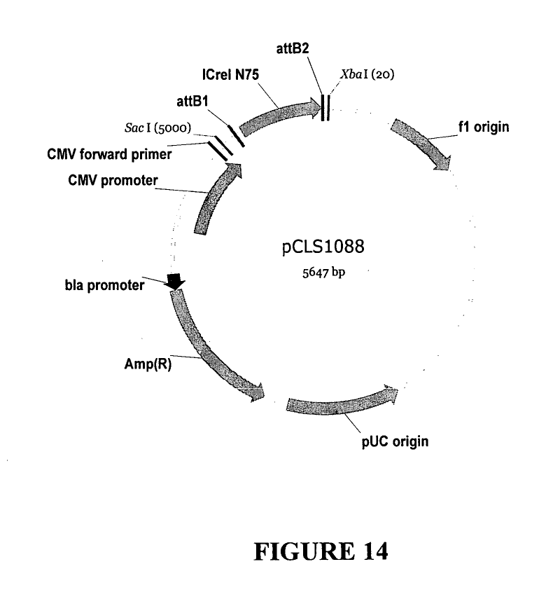

FIG. 14: represents the pCLS1088 vector map.

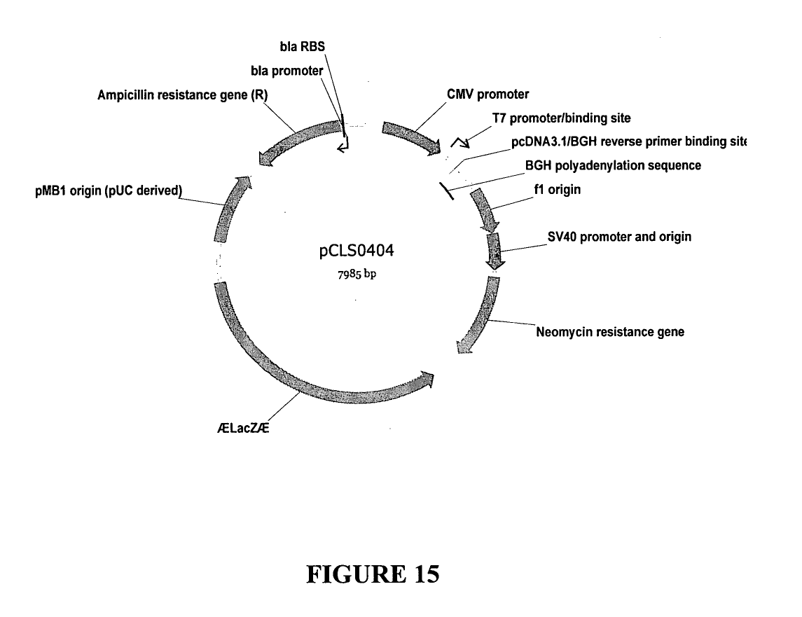

FIG. 15: represents the pCLS0404 vector map

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Making of Meganucleases Cleaving the IL2RG3.6 Target Sequence by Using a Sequential Combinatorial Approach

The IL2RG3.6 target DNA sequence (SEQ ID NO: 7) differs from IL2RG3.4 (SEQ ID NO: 6) only by the four central base pairs that are called 2NN_{—}2NN. IL2RG3.4 carries GTAC as the C1221 target (SEQ ID NO: 8) while IL2RG3.6 has a TCTC sequence like the IL2RG3 target (SEQ ID NO: 86, FIG. 1) and is therefore more difficult to cleave by an I-CreI derived mutant. The Inventors have previously observed that the association of a mutant cleaving a palindromic target with a wild-type 2NN_{—}2NN sequence with a mutant cleaving the other palindromic target will increase the probability of cleavage of the target of interest.

To obtain such an IL2RG3.6 cutter, a strategy based on a sequential combinatorial approach was used. This approach is different from the traditional combinatorial approach used to obtain meganucleases cleaving a modified target.

In these experiments using the sequential combinatorial approach, the Inventors looked first for mutants cleaving the IL2C_P target (SEQ ID NO: 9) (FIG. 1). This palindromic target is identical to the 5AGG_P target but with the bases at positions ±11 and ±7 of the IL2RG3.4 target (FIG. 1). IL2C_P cutters were then chosen to create different mutant libraries degenerated at I-CreI amino acid positions 28, 30, 32 and 33 that were screened using the inventors yeast screening assay (see definitions) against the IL2RG3.4 target.

Instead of combining two mutations sets like in prior art methods, the concept of the sequential combinatorial approach is to fix one mutation set (here mutations allowing for IL2C_P cleavage) before looking for the second mutation set. In this second round of selection, site-directed mutagenesis was performed on the IL2RG3.4 proteins obtained so as to obtain an I-CreI enzyme with cleavage activity toward the IL2RG3.6 target.

Material and Methods

a) Construction of the Sequential Mutant Libraries SeqLib1 and SeqLib2

Using the method according to the present invention, in step a. experiments were conducted to screen 36 I-CreI mutants able to cleave the 5AGG_P target for activity also against the IL2C_P target, this gave some positive clones (FIG. 2). Three positive mutants were isolated and it is these which were used to generate the SeqLib1 and SeqLib2 mutant libraries as detailed below.

The two mutant libraries SeqLib1 and SeqLib2 were generated from the DNA of a pool of the three IL2C_P cutters. To build SeqLib1, which contains mutations at positions 30 and 33, two separate overlapping PCR reactions were carried out that amplify the 5′ end (aa positions 1-41) or the 3′ end (aa positions 34-166) of the I-CreI derived mutants coding sequence. These experiments correspond to step c. of the method according to the present invention and generate the second series of variants which in turn were screened for their activity for the second altered characteristic, in this example IL2RG3.4 cleavage.

The template was the pCLS0542 vector (FIG. 5).

For the 3′ end, PCR amplification is carried out using a primer specific to the pCLS0542 vector (Gal10R 5′-acaaccttgattggagacttgacc-3′; SEQ ID NO: 10) and a primer specific to the I-CreI coding sequence for amino acids 34-43 (10RG34 For 5′-aagtttaaacatcagctaagcttgaccttt-3′; SEQ ID NO: 11). For the 5′ end, PCR amplification is carried out using a primer specific to the pCLS0542 vector (Gal10F 5′-gcaactttagtgctgacacatacagg-3′: SEQ ID NO: 12) and a primer specific to the I-CreI coding sequence for amino acids 25-41 (10RG34Rev1 5′-caagcttagctgatgtttaaacttmnnagactgmnntggtttaatctgagc-3′; SEQ ID NO: 13). The MNN code in the oligonucleotide resulting in a NNK codon at positions 30 and 33 allows the degeneracy at these positions among the 20 possible amino acids. The SeqLib2 library that contains mutations at positions 28, 32 and 33 was built using the same method but with the use of the primer 10RG34Rev2 (5′-caagcttagctgatgtttaaacttmbnmbnctggtttggmbnaatctgagc-3′; SEQ ID NO: 14) instead of 10RG34Rev1. The MBN code in the oligonucleotide resulting in a NVK codon at positions 28, 32 and 33 allows the degeneracy at these positions among all the 20 possible amino acids but F, L, M, I and V. Then, for both libraries, 25 ng of each of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz R D and Woods R A Transformation of yeast by lithium acetate/single-stranded carrier DNA/polyethylene glycol method. Methods Enzymol. 2002; 350:87-96). An intact coding sequence containing mutations at desired positions is generated by in vivo homologous recombination in yeast.

b) Site-Directed Mutagenesis

The I132V and E80K mutations were introduced on a DNA pool consisting of DNA molecules encoding Seq4, Seq5 and Seq7 I-CreI mutants from Table I below. This further modification of the variants isolated in step d. of the method according to the present invention shows that further iterative steps can be used to introduce further altered characteristics into a meganuclease generated according to the method.

Site-directed mutagenesis libraries were created by PCR. For example, to introduce the I132V substitution into the coding sequences of the mutants, two separate overlapping PCR reactions were carried out that amplify the 5′ end (residues 1-137) or the 3′ end (residues 127-167) of the I-CreI coding sequence. For both the 5′ and 3′ end, PCR amplification is carried out using a primer with homology to the vector [Gal10F 5′-gcaactttagtgctgacacatacagg-3′ (SEQ ID NO: 12) or Gal10R 5′-acaaccttgattggagacttgacc-3′ (SEQ ID NO: 10) and a primer specific to the I-CreI coding sequence for amino acids 14-24 that contains the substitution mutation I132VF: 5′-acctgggtggatcaggttgcagctctgaacgat-3′(SEQ ID NO: 22) and I132VR: 5′-atcgttcagagctgcaacctgatccacccaggt-3′(SEQ ID NO: 23).

The resulting PCR products contain 33 by of homology with each other. The PCR fragments were purified. Finally, approximately 25 ng of each of the two overlapping PCR fragments and 75 ng of vector DNA (pCLS1107 (FIG. 12)) were linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the I132V substitution are generated by in vivo homologous recombination in yeast.

The same strategy is used with the following pair of oligonucleotides to create the other libraries containing the E80K substitution:

(SEQ ID NO: 24)
E80KF: 5′-ttaagcaaaatcaagccgctgcacaacttcctg-3′
and
(SEQ ID NO: 25)
E80KR: 5′-caggaagttgtgcagcggcttgattttgcttaa-3′.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, Genetix). Mutants were gridded on nylon filters covering YPD plates, using a low gridding density (about 4 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

d) Sequencing of Mutants

To recover the mutant expressing plasmids, yeast DNA was extracted using standard protocols and used to transform E. coli. Sequence of mutant ORF were then performed on the plasmids by MILLEGEN SA. Alternatively, ORFs were amplified from yeast DNA by PCR (Akada et al., Biotechniques, 2000, 28, 668-670), and sequence was performed directly on PCR product by MILLEGEN SA.

Results

The yeast screening of 36 I-CreI mutants able to cleave the 5AGG_P target against the IL2C_P target gave some positive clones (FIG. 2). Three positive mutants were isolated. They all have the I24V mutation and have respectively the following sequences: TRSER, TYSER, RYSET, where letters indicate amino acids at positions 44, 68, 70, 75 and 77 (for example, TRSER stands for T44, R68, S70, E75 and R77). Using the DNA of these three positive clones toward the IL2C_P target, two different mutant libraries were then built by degenerating amino acids positions 30 and 33 for the first library (SeqLib1) and 28, 32 and 33 for the second library (SeqLib2). Yeast screening of 1116 clones from library 1 against the IL2RG3.4 target yielded 6 positives clones with a unique sequence (FIG. 3) and the screening of 2232 clones from library 2 gave one positive clone. The sequence of the seven IL2RG3.4 cutters is given in Table I.

TABLE I
Sequences of the seven IL2RG3.4 cutters
obtained by a sequential combinatorial
method. Letters indicate amino acids at
positions 28, 30, 32, 33, 38,
40, 44, 68, 70, 75 and77 of I-CreI

| | SEQ | |
|---|---|---|
| | ID | |
| Clones | NO | Sequence |
| Seq1 | 15 | V24-KRSYQS/TRSER |
| Seq2 | 16 | V24-KRSNQS/TYSER |
| Seq3 | 17 | V24-KRSAQS/TRSER |
| Seq4 | 18 | V24-KRSVQS/TRSER + |
| | | Q50R |
| Seq5 | 19 | V24-KRSVQS/RYSET + |
| | | V129A |
| Seq6 | 20 | V24-KRSVQS/TYSER |
| Seq7 | 21 | V24-KNGHQS/TRSER |

As the cleavage activity toward the IL2RG3.4 target for the seven clones Seq1 to Seq7 was still relatively weak, the mutations E80K and I132V were introduced by site-directed mutagenesis on a pool of mutants constituted by the Seq4, Seq5 and Seq7 clones. The screening of the resulting mutants gave very strong cutters against the IL2RG3.4 target and three clones among them with a unique sequence given in Table II were able to cleave the IL2RG3.6 target (FIG. 4).

TABLE II
Sequence of the three IL2RG3.6
cutters. The clones are ranked with
a decreasing cleavage activity

| IL2RG3.6 | SEQ | |
|---|---|---|
| Cutters | ID NO | Sequence |
| Amel1 | 26 | V24-KNGHQS/TRSER + Q50R, |
| | | I132V |
| Amel2 | 27 | V24-KNGHQS/TRSER + E80K |
| Amel3 | 28 | V24-KRSVQS/TRSER + Q50R, |
| | | E80K, V129A |

Therefore using the Sequential Combinatorial Approach, three I-CreI derived mutants able to cleave the IL2RG3.6 have been obtained. The initial IL2RG3.4 cutters have been isolated by using a sequential combinatorial approach, which validates this concept described in the introduction of this example. The three IL2RG3.6 cutters can now be used in co-expression with IL2RG3.3 mutants to cleave the IL2RG3 target. The co-expression of two I-CreI monomers created using the method according to the present invention can be used to generate a hetero-dimeric I-CreI variant in vivo/in vitro. See example 5 below for further details.

### EXAMPLE 2

### Making of new DmoCre Derived Mutants Combining Two Sets of Mutations and Cleaving the Combined DC10TGG4ACT Target

Another strategy to broaden the range of targets recognised and cut by meganucleases is to combine domains from distinct meganucleases. This approach has been used to create new meganucleases by domain swapping between I-CreI and I-DmoI, leading to the generation of a meganuclease cleaving the hybrid sequence corresponding to the fusion of the two half parent target sequences (Epinat et al., Nucleic Acids Res. 2003; 31:2952-62, Chevalier et al., Mol. Cell. 2002; 10:895-905).

The Applicant has previously conducted experiments with its own DmoCre scaffold to seek to broaden the range of DNA target sequences cleaved by engineered homing nuclease enzymes. DmoCre is a chimeric molecule built from the two homing endonucleases I-DmoI and I-CreI. It includes the N-terminal portion from I-DmoI linked to an I-CreI monomer. DmoCre could have a tremendous advantage as scaffold: mutation in the I-DmoI moiety could be combined with mutations in the I-CreI domain, and thousands of such variant I-CreI molecules have already been identified and profiled (Smith J et al., Nucleic Acids Res. 2006; 34(22):e149, Arnould S et al., J Mol. Biol. 2006; 355:443-58, Arnould S et al., J Mol. Biol. 2007; 371:49-65)

The inventors have improved the existing DmoCre scaffold by increasing the overall activity of this enzyme. In particular three mutations were introduced into the I-DmoI N-terminal α-helix of DmoCre corresponding to residues 15, 19 and 20 of I-DmoI (SEQ ID NO: 29). This improved scaffold is known as DmoCre2 (SEQ ID NO: 36).

The possibility of combining different sets of mutations previously isolated for the DmoCre2 protein to cleave a combined target was investigated.

First, eight DmoCre2 derived mutants mutated at residues corresponding to positions 75, 76 and 77 in wild type I-DmoI (SEQ ID NO: 29) and able to cleave the DC4ACT target (SEQ ID NO: 32) were chosen, see Table III for the sequence at residues corresponding to positions 75-77 in SEQ ID NO: 29; these mutants were used to create a mutant library (SeqDC10NNN4ACT) degenerated at DmoCre2 residues corresponding to amino acids positions 29 and 33 in SEQ ID NO: 29. The resulting library was finally screened in yeast against the combined DC10TGG4ACT target (SEQ ID NO: 30).

Material and Methods

Construction of the DC10TGG4ACT Target Vector:

The target was cloned as follows: an oligonucleotide corresponding to the target sequence flanked by gateway cloning sequence was ordered from Proligo: 5′TGGCATACAAGTTTTCCCAGGAAGTTACGACGTTTTGACAATCGTCTGT CA-3′ SEQ ID NO: 31. Double-stranded target DNA, generated by PCR amplification of the single stranded oligonucleotide, was cloned using the Gateway protocol (Invitrogen) into yeast reporter vector (pCLS1055, FIG. 13). Yeast reporter vector was transformed into S. cerevisiae strain FYBL2-7B (MAT a, ura3Δ851, trp1Δ63, leu2Δ1, lys2Δ202).

Construction of the DmoCre2 SeqDC10NNN4ACT Mutant Library:

First, the DNA coding for the eight DmoCre2 mutants able to cleave the DC4ACT target were pooled, these eight mutants were isolated according to steps a. and b. of the method according to the present invention. Then, this DNA pool was used as a template for two separate overlapping PCR reactions in order to generate DmoCre2 derived coding sequences containing mutations at positions 29 and 33, these corresponding to step c. of the method according to the present invention. The first PCR reaction amplifies the 5′ end of DmoCre2 coding sequence (aa positions 1-40) using the primers Gal10F (5′-GCAACTTTAGTGCTGACACATACAGG-3′ SEQ ID NO: 12) and D10CreRev2 (5′-GATCACAACACGATATTCGCTMNNGTTACC TTTMNNTTTCAGCTTGTA-3′ SEQ ID NO: 33) and the second PCR reaction amplifies the 3′ end (positions 34-264) of the DmoCre2 coding sequence using the primers specific Gal10R (5′-ACAACCTTGATTGGAGACTTGACC-3′ SEQ ID NO: 10) and D10CreFor2 (5′-AGCGAATATCGTGTTGTGATCACCCAGAAGTCTG-3′ SEQ ID NO: 35).

The MNN code in the D10CreRev2 oligonucleotide resulting in a NNK codon at positions 29 and 33 allows the degeneracy at these positions among the possible amino acids. Then, 25 ng of each of the two overlapping PCR fragments and 75 ng of overlapping vector DNA (pCLS0542, FIG. 5) linearized by digestion with NcoI and EagI were used to transform the yeast Saccharomyces cerevisiae strain FYC2-6A (MAT a, ura3Δ851, trp1Δ63, leu2Δ1, lys2Δ202) using a high efficiency LiAc transformation protocol (Gietz et al., Methods Enzymol. 2002; 350:87-96). An intact coding sequence was generated by in vivo homologous recombination in yeast. After transformation, 2232 clones of the SeqDC10NNN4ACT library were picked.

Mating of Meganuclease Expressing Clones and Screening in Yeast:

Screening was performed as described previously (Arnould et al., J Mol Biol. 2006; 355:443-58). Specifically, mating was performed using a colony gridder (QpixII, Genetix). Mutants were gridded on nylon filters covering YPD plates, using a low gridding density (about 4 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using proprietary software.

Sequencing of Mutants

To recover the mutant expressing plasmids, yeast DNA was extracted using standard protocols and used to transform E. coli. Sequencing of mutant ORF were then performed on the plasmids by Millegen SA. Alternatively, ORFs were amplified from yeast DNA by PCR (Akada et al., Biotechniques. 2000; 28:668-70, 672, 674), and sequencing was performed directly on PCR product by Millegen SA.

Results

Eight DmoCre2 derived mutants able to cleave the DC4ACT target (SEQ ID NO: 32) were chosen. These mutants carry mutations at residues corresponding to positions 75, 76 and 77 in SEQ ID NO: 29 and are listed in Table III below.

TABLE III
Sequence (aa 75 to 77) of the eight
DC4ACT cutters that were chosen to
create the SeqDC10NNN4ACT library

| Mutant | SEQ | Sequence, aa |
|---|---|---|
| Name | ID NO | 75 to 77 |
| Mt1-DC4ACT | 37 | RSV |
| Mt2-DC4ACT | 38 | HSC |
| Mt3-DC4ACT | 39 | NGA |
| Mt4-DC4ACT | 40 | HTS |
| Mt5-DC4ACT | 41 | RTV |
| Mt6-DC4ACT | 42 | ATN |
| Mt7-DC4ACT | 43 | CTC |
| Mt8-DC4ACT | 44 | TTV |

The SeqDC10NNN4ACT library was then screened using our yeast screening assay toward the combined DC10TGG4ACT target. The screening assay gave 11 positive clones and part of the screening is shown in FIG. 6, where three positive clones are black circled. Thus, the inventors have shown that it is possible to associate mutations of residues interacting with nucleotides at positions +8 to +10 of the C12D34 target (SEQ ID NO: 34) with mutations of residues interacting with nucleotides at positions +2 to +4 of the C12D34 target in order to cleave a combined target.

The sequences of the 11 positives clones gave nine unique sequences which are listed in the Table IV below.

TABLE IV
List of nine unique sequences for the eleven positive
DC10TGG4ACT target cutters.

| | | Sequences (Mutations in |
|---|---|---|
| DC10TGG4ACT | | comparison to the DmoCre2 |
| Cutters | SEQ ID NO | protein (SEQ ID NO: 29) are indicated) |
| 1 | 54 | 33T75R77V |
| 2 | 55 | 29H33T75R77V |
| 3 | 56 | 29K33I75R77V |
| 4 | 57 | 29F33I75H76S77C |
| 5 | 58 | 29H33I75H76S77C |
| 6 | 59 | 33I75C77C |
| 7 | 60 | 29F33I75R77V |
| 8 | 61 | 29H33L75R77V |
| 9 | 62 | 29K33V75R77V |

### EXAMPLE 3

### Making of Meganucleases Cleaving the IL2RG3.3 and IL2RG3.5 Target Sequences by Using a Sequential Combinatorial Approach

To obtain IL2RG3.3 cutters, a sequential combinatorial approach was adopted. In a prior art approach, to cleave such a target, mutations from 5CTG_P cutters and 10GAC_P cutters would be combined to cleave the combined IL2RG3.3 target (SEQ ID NO: 88).

Using instead the sequential combinatorial approach detailed in the present patent application, three 5CTG_P cutters (representing the selected variant of step b.) were chosen to create three different mutant libraries that were screened in yeast for cleavage activity toward the IL2RG3.3 and IL2RG3.5 target (representing the selected variants of step d.). The IL2RG3.5 DNA sequence differs only from IL2RG3.3 by the four central base pairs that are called 2NN_{—}2NN. IL2RG3.3 carries GTAC as the C1221 target while IL2RG3.5 has a TCTC sequence like the IL2RG3 target and is therefore more difficult to cleave by an I-CreI derived mutant. Activity toward the IL2RG3.5 target was then enhanced by site directed mutagenesis on proteins obtained by the sequential combinatorial method.

Material and Methods

a) Construction of the sequential mutant libraries Seq10IL2RG3-1, SeqIL2RG3-2 et Seq10IL2RG3-3

The three mutant libraries Seq10IL2RG3-1, SeqIL2RG3-2 and Seq10IL2RG3-3 were generated from the DNA of a pool of three 5CTG_P cutters. To build Seq10IL2RG3-1, which contains mutations at positions 30 and 33, two separate overlapping PCR reactions were carried out that amplify the 5′ end (aa positions 1-39) or the 3′ end (aa positions 34-166) of the I-CreI derived mutants coding sequence. For the 3′ end, PCR amplification is carried out using a primer specific to the pCLS0542 vector (Gal10R 5′-ACAACCTTGATTGGAGACTTGACC-3′ (SEQ ID NO: 10)) and a primer specific to the I-CreI coding sequence for amino acids 34-43 (10RG33For1 5′-aagtttaaacatcagctaagcttgaccttt-3′ (SEQ ID NO: 45)). For the 5′ end, PCR amplification is carried out using a primer specific to the pCLS0542 vector (Gal10F 5′-GCAACTTTAGTGCTGACACATACAGG-3′ (SEQ ID NO: 12)) and a primer specific to the I-CreI coding sequence for amino acids 25-39 (10RG33Rev1 5′-tagctgatgtttaaacttmnnagactgmnntggtttaatctgagc-3′ (SEQ ID NO: 46)). The MNN code in the oligonucleotide resulting in a NNK codon at positions 30 and 33 allows the degeneracy at these positions among the 20 possible amino acids.

The SeqIL2RG3-2 library that contains mutations at positions 33 and 40 was built using the same method but with the use of the primer 10RG33For2 (5% aagtttaaacatcagctannkttgaccttt-3′ (SEQ ID NO: 47)) instead of 10RG33For1 and primer 10RG33Rev2 (5′-tagctgatgtttaaacttmnnagactggtttggtttaatctgagc-3′ (SEQ ID NO: 48)) instead of 10RG33Rev1. For the third Seq10IL2RG3-3 library that degenerate residues 28, 33 and 40, the primers 10RG33For3 (5′-aagtttaaacatcagctanvkttgaccttt-3′ (SEQ ID NO: 49)) and 10RG33Rev3 (5′-tagctgatgtttaaacttmbnagactggtttggmbnaatctgagc-3′ (SEQ ID NO: 50)) were used.

The NVK codon at positions 28, 33 and 40 allows the degeneracy at these positions among all the 20 possible amino acids but F, L, M, I and V. Then, for both libraries, 25 ng of each of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leu2Δ1, h is 3Δ200) using a high efficiency LiAc transformation protocol (Gietz R D and Woods R A Transformation of yeast by lithium acetate/single-stranded carrier DNA/polyethylene glycol method. Methods Enzymol. 2002; 350:87-96). An intact coding sequence containing mutations at desired positions is generated by in vivo homologous recombination in yeast. 1116 clones were picked for the two first libraries and 2232 for the third library.

b) Site-Directed Mutagenesis

The F54L, Y66H, E80K, V105A and I132V mutations were introduced on a DNA pool constituted by the SeqA, SeqB and SeqC I-CreI mutants, SEQ ID NO: 51 to 53 respectively, see Table IV below.

c) Mating of Meganuclease Expressing Clones and Screening in Yeast Sequencing of Mutants

Performed as in example 1 above.

d) Sequencing of Mutants

Performed as in example 1 above.

Results

Three I-CreI derived mutants were chosen for their ability to cleave the 5CTG_P target. They carry respectively the following mutations in comparison to the wild-type I-CreI enzyme: 24V44R68Y70S75E77R (SEQ ID NO: 105), 44K68Y70S75E77V (SEQ ID NO: 106) and 44R68Y70S77N (SEQ ID NO: 107). Using the DNA of these three mutants, three different mutant libraries were then built by degenerating amino acids positions 30 and 33 for the first library (Seq10IL2RG3-1), 33 and 40 for the second library (Seq10IL2RG3-2) and 28, 33 and 40 for the third library (Seq10IL2RG3-3). These three mutant libraries were screened toward both targets (IL2RG3.3 and IL2RG3.5) using our yeast screening assay. FIG. 7 shows an example of this assay. The screen yielded a lot of positive clones toward the IL2RG3.3 target and just some of them (18 out of 345, 5%) were also able to cleave the IL2RG3.5 target, but more weakly than the IL2RG3.3 target. Almost all the IL2RG3.5 cutters belong to the Seq10IL2RG3-2 library

To enhance cleavage activity toward the IL2RG3.5 target, three IL2RG3.5 cutters (SeqA, SeqB and SeqC) were then chosen and the mutations F54L, Y66H, E80K, V105A and I132V were respectively introduced on a DNA pool constituted by the three mutants, whose sequence is indicated in the table V below.

TABLE V
Sequence of the three IL2RG3.5 cutters obtained by the sequential
approach and that were selected for further activity improvement.

| | SEQ | |
|---|---|---|
| Mutant | ID | Sequence (mutations in comparison to |
| Name | NO: | wild-type I-CreI sequence) |
| SeqA | 51 | 33H44K70S75E77V |
| SeqB | 52 | 30R33R44K68Y70S75E77V |
| SeqC | 53 | 33H40C44R68Y70S75E77R |

The screening of the resulting mutants gave very strong cutters against the IL2RG3.5 target as shown in FIG. 8. In conclusion, the sequential combinatorial approach has allowed the inventors to obtain strong IL2RG3.3 cutters with some of them able to cleave the IL2RG3.5 target. Activity toward the IL2RG3.5 target could be further refined to obtain saturating cleavage activity in yeast.

### EXAMPLE 4

### Making of Meganucleases Cleaving the IL2RG3 Target by Co-Expression of IL2RG3.3 and IL2RG3.4 Mutants Obtained Both by the Sequential Combinatorial Method

In order to determine whether I-CreI monomers generated using the sequential combinatorial approach could be used as the components of a heterodimeric I-CreI enzyme able to cleave a new target, the Inventors decided to test whether the IL2RG3.3 and IL2RG3.4 mutants obtained using the sequential combinatorial approach in examples 1 and 3 above could be coexpressed and whether the resulting heterodimer was able to cleave the combined IL2RG3 target.

The IL2RG3.3 and IL2RG3.4 mutants, were coexpressed in yeast. The co-expression lead to the formation of heterodimers, whose activity toward the IL2RG3 target was monitored.

Material and Methods

Recloning of IL2RG3.4 Cutters into the pCLS1107 Vector

To coexpress two mutants in yeast, each mutant must be in a different vector backbone with a different selection marker. For that purpose, IL2RG3.4 mutants were cloned into the pCLS1107, see FIG. 12. A PCR was performed using the Gall OF (SEQ ID NO: 12) and Gal10R (SEQ ID NO: 10) primers. 25 ng of each of the PCR fragment and 75 ng of vector DNA (pCLS1107) linearized by digestion with NgoMIV and DraIII were used to transform the yeast Saccharomyces cerevisiae strain FYC2-6A (MATα, trp1Δ63, leu2Δ1, his3Δ200) using a high efficiency LiAc transformation protocol.

Results

Among the IL2RG3.3 cutters, nine IL2RG3.5 cutters were coexpressed with seven IL2RG3.4 cutters (three of them cleaving the IL2RG3.6 target). The activity of the 63 resulting heterodimers was monitored on the IL2RG3 target using the yeast screening assay described previously in examples 1, 2 and 3.

TABLE VI
IL2RG3.5 mutants that were chosen for co-expression
to cleave the IL2RG3 target.

| IL2RG3.5 | SEQ | |
|---|---|---|
| Cutter Name | ID NO | IL2RG3.5 Cutter Sequence |
| SeqIL2RG5-1 | 63 | 24V33H40C44R50R68Y70S75E- |
| | | 77R |
| SeqIL2RG5-2 | 64 | 24V33H40C44R54L68Y70S75E- |
| | | 77R |
| SeqIL2RG5-3 | 65 | 24V33H44K66H68Y70S75E77V |
| SeqIL2RG5-4 | 66 | 24V30R33R44K66H68Y70S75E- |
| | | 77V |
| SeqIL2RG5-5 | 67 | 24V33H44K68Y70S75E77V80K |
| SeqIL2RG5-6 | 68 | 24V33H40C44R68Y70S75E77R- |
| | | 80K |
| SeqIL2RG5-7 | 69 | 24V30R33R44K68Y70S75E77V- |
| | | 105A |
| SeqIL2RG5-8 | 70 | 24V30R33R44K68Y70S75E77V- |
| | | 132V |
| SeqIL2RG5-9 | 71 | 24V33H44K68Y70S75E77V132V |

TABLE VII
IL2RG3.4 mutants that were chosen for co-expression
to cleave the IL2RG3 target.

| IL2RG3.4 | SEQ | |
|---|---|---|
| Cutter Name | ID NO | IL2RG3.4 Cutter Sequence |
| SeqIL2RG4-1 | 72 | 24V32G33H44T54V68Y70S75E77R129A |
| SeqIL2RG4-2 | 73 | 24V32G33H44T50R66H68Y70S75E77R129A |
| SeqIL2RG4-3 | 74 | 24V30R33V44T50R70S75E77R80K |
| SeqIL2RG4-4 | 75 | 24V30R33V44T50R68Y70S75E77R80K129A |
| SeqIL2RG4-5 | 76 | 24V32G33H44T50R70S75E77R80K |
| SeqIL2RG4-6 | 77 | 24V30R33V44T50R68Y70S75E77R132V |
| SeqIL2RG4-7 | 78 | 24V32G33H44T50R68Y70S75E77R132V |

FIG. 9 shows that almost all the different 63 heterodimers cleave the IL2RG3 target with different cleavage intensities. Some heterodimers achieve IL2RG3 cleavage with a strong intensity.

In conclusion, mutants obtained using the combinatorial approach have led to a wide selection of heterodimeric I-CreI enzymes which exhibit good levels of IL2RG3 cleavage in yeast.

### EXAMPLE 5

### Induction of Gene Correction Activity in CHO Cells with the IL2RG3 Meganuclease Formed by Co-Expression of Mutants Obtained by the Sequential Combinatorial Method

The IL2RG3 meganuclease formed by the modified I-CreI monomers of SeqIL2RG5-1 (SEQ ID NO: 63) and the SeqIL2RG4-7 (SEQ ID NO: 78), one of the 63 heterodimers tested in example 4, was then checked for its ability to induce gene correction in CHO cells.

This assay is based on the use of a chromosomal reporter system in CHO cells (FIG. 10). In this system a single-copy of the LacZ gene is driven by the CMV promoter is interrupted by the IL2RG3 sequence and is as a result non-functional. The transfection of the CHO cell line with plasmids coding for both partners of the IL2RG3 meganuclease and a LacZ repair plasmid allows the restoration of a functional LacZ gene by homologous recombination.

It has previously been shown that double-strand breaks can induce homologous recombination; therefore the frequency with which the LacZ gene is repaired is indicative of the cleavage efficiency of the genomic IL2RG3 target site.

Material and Methods

a) Cloning of the SeqIL2RG5-1 and SeqIL2RG4-7 I-CreI Derived Mutants into a Mammalian Expression Vector

Each mutant ORF was amplified by PCR using the primers CCM2For (5′-aagcagagctctctggctaactagagaacccactgcttactggcttatcgaccatggccaataccaaatataacaaagagttcc-3′: SEQ ID NO: 100) and CCMRev60 (5′-ctgctctagactaaggagaggacttfttcttctcag-3′: SEQ ID NO: 101). The PCR fragment was digested by the restriction enzymes Sad and XbaI, and was then ligated into the vector pCLS1088 (FIG. 14) digested also by Sad and XbaI. Resulting clones were verified by sequencing (MILLEGEN).

b) Chromosomal Assay in CHO-K1 Cells

CHO-K1 cell lines harbouring the reporter system were seeded at a density of 2×10⁵ cells per 10 cm dish in complete medium (Kaighn's modified F-12 medium (F12-K), supplemented with 2 mM L-glutamine, penicillin (100 UI/ml), streptomycin (100 μg/ml), amphotericin B (Fongizone) (0.25 μg/ml) (INVITROGEN-LIFE SCIENCE) and 10% FBS (SIGMA-ALDRICH CHIMIE). The next day, cells were transfected with Polyfect transfection reagent (QIAGEN). Briefly, 2 μg of lacz repair matrix vector (pCLS0404, FIG. 15) was co-transfected with various amounts of meganucleases expression vectors.

After 72 hours of incubation at 37° C., cells were fixed in 0.5% glutaraldehyde at 4° C. for 10 min, washed twice in 100 mM phosphate buffer with 0.02% NP40 and stained with the following staining buffer (10 mM Phosphate buffer, 1 mM MgCl₂, 33 mM K hexacyanoferrate (III), 33 mM K hexacyanoferrate (II), 0.1% (v/v) X-Gal). After, an overnight incubation at 37° C., plates were then examined under a light microscope and the number of LacZ positive cell clones counted. The frequency of LacZ repair is expressed as the number of LacZ+ foci divided by the number of transfected cells (5×10⁵) and taking into account the transfection efficiency.

Results

FIG. 10 shows that the meganuclease formed by the co-expression of the SeqIL2RG5-1 and SeqIL2RG4-7 mutants is able to induce gene correction at a frequency level of 0.3% in CHO cells.

This same experiment has been conducted with meganucleases constituted by I-CreI derived mutants issued from the classical combinatorial method but gene correction frequency of only 0.05% could have been obtained. Therefore, the sequential combinatorial method has yielded meganucleases able to cleave the IL2RG3 target and that can induce gene correction in CHO cells at higher levels than the meganucleases obtained by the classical combinatorial method.

### EXAMPLE 6

### Making of Meganucleases Cleaving the B2M11.4 Target Sequence by Using a Sequential Combinatorial Approach

Beta-2-microglobulin (B2M) is a serum protein found in association with the major histocompatibility complex (MHC) class I heavy chain on the surface of nearly all nucleated cells. The 22 bp B2M11 DNA sequence (5′-TGAAATTAGGTACAAAGTCAGA-3′ (SEQ ID NO: 98)) is located in the first intron of the human B2M coding gene. The B2M11.4 target (5′-TCTGACTTTGTACAAAGTCAGA-3′ (SEQ ID NO: 99)) is a palindromic target derived from the right part of the B2M11 DNA sequence (FIG. 11a).

Using prior art classical methods, the inventors have obtained only one very weak B2M11.4 cutter.

Therefore, in order to find new I-CreI derived mutants able to cleave the B2M11.4 target, a sequential combinatorial process was undertaken. The B2M11.4 target is a combination of the 5TTT_P and 10CTG_P targets. Therefore, nine I-CreI mutants able to cleave the 5TTT_P target (isolated according to steps a. and b. of the method according to other present invention) were selected and the Seq10B2M11.4 mutant library degenerated at positions 33 and 38 was built and screened in the yeast (corresponding to steps c. and d. of the method according to the present invention). The initial B2M11.4 cleavage activity of the selected variants was then further enhanced by site-directed mutagenesis.

Material and Methods

The Seq10B2M11.4 mutant library was generated from the DNA of a pool of nine 5TTT_P cutters. To build Seq10B2M11.4, which contains mutations at positions 33 and 38, two separate overlapping PCR reactions were carried out that amplify the 5′ end (aa positions 1-32) or the 3′ end (aa positions 27-166) of the I-CreI derived mutants coding sequence. For the 3′ end, PCR amplification is carried out using a primer specific to the pCLS0542 vector (Gal10R 5′-ACAACCTTGATTGGAGACTTGACC-3′ (SEQ ID NO: 10)) and a primer specific to the I-CreI coding sequence for amino acids 27-41 (Seq10BMFor1 5′-attaaaccaaaccagtctnvkaagtttaaacatnvkctaagcttg-3′ (SEQ ID NO: 102)). For the 5′ end, PCR amplification is carried out using a primer specific to the pCLS0542 vector (Gal10F 5′-GCAACTTTAGTGCTGACACATACAGG-3′ (SEQ ID NO: 12)) and a primer specific to the I-CreI coding sequence for amino acids 25-32 (Seq10BMRev1 5′-agactggtttggtttaatctgagc-3′ (SEQ ID NO: 103)). The NVK codon at positions 33 and 38 allows the degeneracy at these positions among all the 20 possible amino acids but F, L, M, I and V. Then, 25 ng of each of the two overlapping PCR fragments and 75 ng of vector DNA (pCLS1107) linearized by digestion with NgoMIV and DraIII were used to transform the yeast Saccharomyces cerevisiae strain FYC2-6A (MATα, trp1Δ63, leu2Δ1, his3Δ200) using a high efficiency LiAc transformation protocol (Gietz R D and Woods R A Transformation of yeast by lithium acetate/single-stranded carrier DNA/polyethylene glycol method. Methods Enzymol. 2002; 350:87-96). An intact coding sequence containing mutations at desired positions is generated by in vivo homologous recombination in yeast. 2232 yeast clones were then picked to constitute the library.

Results

Nine I-CreI derived mutants able to cleave the 5TTT_P target, whose sequences are listed in the Table VIII below, were used to create the Seq10B2M11.4 mutant library.

TABLE VIII
Sequence of the nine 5TTT_P cutters used to
create the Seq10B2M11.4 library. The sequences
of the mutants (otherwise identical to I-CreI)
are given by the nature of amino acid
at positions 44, 68, 70, 75 and 77.

| | SEQ | Sequence at |
|---|---|---|
| | ID | positions 44, 68, |
| 5TTT_P Cutters | NO | 70, 75 and 77 |
| 1 | 89 | QRRNK |
| 2 | 90 | QNSNI |
| 3 | 91 | KYSNI + V24 |
| 4 | 92 | QASNR |
| 5 | 93 | QRSNI |
| 6 | 94 | KNNDI |
| 7 | 95 | KRTDI |
| 8 | 96 | KTTDI |
| 9 | 97 | KRNDI |

The Seq10B2M11.4 mutant library was screened against the B2M11.4 target using our yeast screening assay. Only one very weak cutter (SeqB2M11.4 Mutant) with detectable activity was identified. Its sequence is 31R33G38Y44K70T (SEQ ID NO: 108). It appears to be derived from the KRTDI 5TTT_P cutter (SEQ ID NO: 95) with mutations 33G38Y introduced during the library construction and the 31R coming probably from a PCR mutation. To enhance B2M11.4 cleavage activity, the mutations F54L (SEQ ID NO: 109), E80K (SEQ ID NO: 110), V105A (SEQ ID NO: 111) and I132V (SEQ ID NO: 112) were introduced individually by site-directed mutagenesis in the SeqB2M11.4 mutant and activity of the resulting proteins was monitored in yeast toward the B2M11.4 target. FIG. 11b shows the SeqB2M11.4 mutant with the F54L or I132V mutation presents a good cleavage activity toward the B2M11.4.

In conclusion, the sequential combinatorial method is a better alternative to the prior art method to obtain I-CreI mutants with modified specificity and able to cleave the target of interest.

1. A method to generate and select a meganuclease having at least two altered characteristics in comparison to a parent meganuclease, comprising:
a. constructing from a parent meganuclease, a first series of variants which differ from said parent meganuclease by at least one acid amino substitution;
b. screening the first series of variants of a. and selecting those which have a first altered characteristic, to obtain selected variants;
c. constructing from the selected variants of b. a second series of variants having a least one other amino acid substitution;
d. screening the second series of variants of b. and selecting those which have said first altered characteristic and a second altered characteristic.

2. The method of claim 1, wherein said parent meganuclease is either a wild type meganuclease or a functional variant of a wild type meganuclease.

3. The method of claim 1, wherein said parent meganuclease of the constructing a. or said selected variants of the constructing c. are a single-chain meganuclease.

4. The method of claim 1, wherein said parent meganuclease is selected from the group consisting of: I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu 1, PI-Mxe I, PI-Npu 1, PI-Pfu I, PI-Rma I, PI-Spb I, PI-Ssp I, PI-Fac I, PI-Mja I, PI-Pho I, PI-Tag I, PI-Thy I, PI-Tko I, PI-Tsp I, I-MsoI, and I-DmoI.

5. The method claim 1, wherein said parent meganuclease comprises at least one I-CreI monomer.

6. The method of claim 5, wherein said at least one I-CreI monomer is modified in at least one selected from the group consisting of the constructing a. and the constructing c., such that at least one of the residues in positions 19, 24, 28, 30, 32, 33, 37, 38, 40, 44, 50, 54, 66, 68, 70, 75, 77, 79, 80, 81, 105, 129, and 132 of said I-CreI monomer is substituted.

7. The method of claim 1, wherein said parent meganuclease is chimeric comprising a first domain from a first meganuclease and a second domain from a second meganuclease.

8. The method of claim 7, wherein said first domain is from I-DmoI.

9. The method of claim 8, wherein said I-DmoI is modified in at least one selected from the group consisting of the constructing a. and the constructing c. of said method, such that at least one of residues in position 4, 15, 19, 20, 27, 29, 33, 35, 37, 49, 52, 75, 76, 77, 81, 92, 94, 95 101, 102, and 109 of said first I-DmoI domain is substituted.

10. The method of claim 1, wherein said at least two altered characteristics are selected from the group consisting of an altered DNA target specificity for at least one nucleotide in said DNA target, altered enzymatic activity levels, altered kinetics, and altered domain-domain structure.

11. The method of claim 1, wherein at least one selected from the group consisting of said first series of variants and said second series of variants are obtained by constructing a nucleic acid library encoding said parent meganuclease of the constructing a. or encoding the selected variants of the screening b. respectively; and
mutating said nucleic acid libraries so as to introduce a mutation into the sequence encoded therein; and
expressing at least one selected from the group consisting of the first series of variants and the second series of variants from each of said respective libraries for screening in b. and d., respectively.

12. The method of claim 11, wherein the library of nucleic acid molecules are created in at least one selected from the group consisting of a. and c. by random mutagenesis of nucleic acid molecules encoding said parent meganuclease.

13. The method according to claim 11, wherein the library of nucleic acid molecules are created in at least one selected from the group consisting of a. and c. by site directed mutagenesis.

14. The method of claim 1, wherein c. and d. are repeated a number of ‘n’ times to generate a meganuclease with a number of ‘n’ additional altered characteristic(s).

15. A polypeptide obtained by the method of claim 1, comprising any one of SEQ ID NO: 51, 52, 53, 54, 55, 56, 57, 58, 59, 60, 61, 62, 63, 64, 65, 66, 67, 68, 69, 70, 71, 72, 73, 74, 75, 76, 77, 78, 89, 90, 91, 92, 93, 94, 94, 95, 96, 97, 105, 106, 107, 108, 109, 110, and 112.

16. A polypeptide obtained by the method of claim 1, consisting of any one selected from the group consisting of SEQ ID NO: 51, 52, 53, 54, 55, 56, 57, 58, 59, 60, 61, 62, 63, 64, 65, 66, 67, 68, 69, 70, 71, 72, 73, 74, 75, 76, 77, 78, 89, 90, 91, 92, 93, 94, 94, 95, 96, 97, 105, 106, 107, 108, 109, 110, and 112.

17. The method of claim 2, wherein said parent meganuclease of the constructing a. or said selected variants of the constructing c. are a single-chain meganuclease.

18. The method of claim 2, wherein said parent meganuclease is selected from the group consisting of: I-Sce I, I-Chu 1, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav 1, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy 1, PI-Tko I, PI-Tsp I, I-MsoI, and I-DmoI.

19. The method of claim 3, wherein said parent meganuclease is selected from the group consisting of: I-Sce I, I-Chu I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu 1, PI-Rma I, PI-Spb I, PI-Ssp I, PI-Fac 1, PI-Mja I, PI-Pho I, PI-Tag I, PI-Thy I, PI-Tko I, PI-Tsp I, I-MsoI, and I-DmoI.

20. The method of claim 2, wherein said parent meganuclease comprises at least one I-CreI monomer.
